(19) United States
(12) Patent Application Publication
(10) Pub. No.: US 2001/0053362 A1
(43) Pub. Date: Dec. 20, 2001

(54) Applications of immune system tolerance to treatment of various diseases

(76) Inventor: Lee Walters, San Diego, CA (US)

Correspondence Address: BROWN, MARTIN, HALLER & McCLAIN, LLP, 1660 UNION STREET, SAN DIEGO, CA, 92101-2926, US

(21) Appl. No.: 09/802,350

(22) Filed: Mar. 9, 2001

Related U.S. Application Data
(60) Provisional application No. 60/188,124, filed on Mar. 9, 2000.

(51) Int. Cl. A61K 39/00

(52) U.S. Cl. 424/184.1; 424/93.7

(57) ABSTRACT

A new approach to immune system transplantation and other organ transplantation is described below. The invention describes the novel use of human tissues, or products derived from human tissues including but not limited to antigens, proteins, glycoproteins, and carbohydrates, taken from an individual patient, group or group of patients, to induce immunological tolerance to human antigens in other mammals. Mammals thus rendered tolerant to human antigens can subsequently serve as immune system donors, and as donors of other biological systems, to recipient humans. The invention also uniquely integrates experimentally documented observations from diverse fields of biological and medical research. The invention provides novel treatments for all cancers; for hereditary and acquired immunodeficiency disorders including AIDS; for failures of host immunological defenses including infectious diseases; for hereditary end acquired bone marrow failure syndromes; and for autoimmune diseases. In addition, the invention provides a novel method for achieving successful organ transplantation in humans, without graft rejection or graft-versus-host disease.

### FIELD OF THE INVENTION

A new approach to immune system transplantation and other organ transplantation is described below. The invention describes the novel use of antigens derived from human tissues, or products derived from human tissues including but not limited to proteins, glycoproteins, and carbohydrates, taken from an individual patient, group or group of patients, to induce immunological tolerance to human antigens in other mammals. Mammals thus rendered tolerant to human antigens can subsequently serve as immune system donors, and as donors of other biological systems, to recipient humans. The invention also uniquely integrates experimentally documented observations from diverse fields of biological and medical research. The invention provides novel treatments for all cancers; for hereditary and acquired immunodeficiency disorders including AIDS; for failures of host immunological defenses including infectious diseases; for hereditary end acquired bone marrow failure syndromes: and for autoimmune diseases. In addition, the invention provides a novel method for achieving successful organ transplantation in humans, without graft rejection or graft-versus-host disease.

### BACKGROUND OF THE INVENTION

Immune system transplantation (also referred to as bone marrow transplantation or hematopoietic stem cell transplantation) is an established medical therapy that can be successfully performed only if the complicating problems of graft vs. host disease and graft rejection can be avoided or successfully treated (see, e.g., Janeway, et al., Immunobiology (1999) Garland Publishing; Brenner, M. K., Cecil Textbook of Medicine, (2000) W. B. Saunders). When these complicating problems are avoided and immune system transplants are successful, this occurs in spite of the fact that modern science has not yet completely described how the various elements of the immune system (including, but not limited to the bone marrow, T and B cells, thymus, and lymphoid tissue such as occur in lymph nodes) function and interact. In current medical practice, the avoidance of graft vs. host disease (GVHD) and graft rejection only occurs when an identical twin acts as the immune system donor. Without an available identical twin, these complications can occur and must be treated. Treatment, often difficult to achieve, is associated with significant morbidity and mortality.

The principal problems associated with organ transplantation are immune rejection and a shortage of acceptable donors. Unless the donor is an identical twin, the immune system of the recipient recognizes the graft as foreign and the recipient's immune system tries to reject the graft. Although immune suppression may postpone rejection for prolonged periods, immune suppression places the recipient at risk for infections and malignancies. Despite requiring chronic immune suppression, most organ and tissue transplants are successful in saving lives and improving the quality of life. The list of successfully transplanted tissues includes: kidney, heart, lung, liver, corneas, pancreas, pancreatic islets of Langerhans, intestines, brain tissue, liver, spleen, thymus, lymph nodes, bone marrow, skin, and bones. Combinations of tissue have also been transplanted; for example, heart-lung transplants, pancreas-kidney transplants, and pancreas-kidney-intestinal transplants.

Immunological tolerance can be induced to molecules that are normally antigenic by exposing the immune system to the molecules while the immune system is still immature (during fetal development or during the neonatal period, depending upon the species and the antigens) (see, e.g., Traub, E., J. Exp Med. (1938) 68:229-50; Nossal, G. J. V., Ann. Rev. Immunol. (1983) 1:33-62; and Billingham et al., Nature (1953) 172:603-6). Both fetal immunization and in utero exposure to antigen can result in a state of immunologic tolerance in the neonate. Tolerance induction of fetal and premature infant lymphocytes has become a paradigm for neonatal responsiveness (see, e.g., Bona & Bot, Immunologist (1997) 5:5-9; Owen, R. D. Proc. R. Soc. Lond. Bull. 146:8-18 (1957).

It has been demonstrated that the immune system of an immunologically deficient or compromised mammal can be reconstituted and functionally repaired with immune or hematopoietic stem cells from a different mammalian species (see, e.g., McCune et al., Science (1988) 241:1632-39). Specifically, the immune system of an immunologically deficient mouse was reconstituted and repaired with fetal human immune or stem cells. The implications of those observations for this invention are several fold; (1) the observations demonstrate that tolerance in one species can be simultaneously induced to a large number of antigens from a different species (xenogeneic antigens). As cited in McCune et al., human fetal immune cells accepted mouse tissue antigens as “self”. (2) The observations demonstrate that the immune system of one species (in this case, human) can survive and function in an animal host of a different species (mouse). In other words, xenogeneic immunological reconstitution is plausible. These observations have also been demonstrated in other animal systems (see, e.g., Mosier, D. E., Nature (1988) 336:256-59; Lubine et al., Science (1991) 252:427-31).

The feasibility of intrauterine antigen introduction and stable chimera production has been demonstrated (see, e.g., Borzy et al., Am. J. Med. Gen. (1984) 18:527-39; Alberts et al., Molecular Biology of the Cell (1995) Garland Publishing, 3^{rd} ed.)

Because of the relative success of the above organ and tissue transplants, a marked shortage of human organ donors exists. For example, although nearly 9,500 kidney transplants are performed annually in the United States, approximately 40,000 Americans develop end stage renal disease annually, and these 40,000 Americans could benefit from organ transplants. Xenografts, herein defined as transplants from another species, could potentially resolve the shortage of transplantable organs and tissues, but the risk of rejection is considered to be even greater than for allografts, herein defined as transplants from a non-identical donor of the same species.

Over the last two decades, organ transplantation has become a routine therapeutic option for patients with end-stage organ failure. Both short-term and long-term outcomes after organ transplantation have improved considerably (Hariharan et al., New Engl. J. Med.(2000) 342:605-12); nevertheless, long-term morbidity and mortality still remain substantial problems. The chronic immunosuppression that organ transplant recipients require for the rest of their lives frequently fails to prevent graft loss due to chronic rejection and is associated with severe side effects, including infections, malignancies, nephrotoxicity, and metabolic disorders. Furthermore, the dramatic shortage of available human organs has renewed interest in the use of organs from other species. The formidable immunological barriers posed by xenotransplantation (Auchincloss HA. Xeno 1995. 3:19-22; Steele & Auchincloss, Annu. Rev. Med. (1995) 46:345-60, Buhler et al., Frontiers in Bioscience (1999) 4:416-32), however, would probably require unacceptably high levels of chronic nonspecific immunosuppression (Zaidi et al., Transplantation (1998) 65:1584-90), which has been avoided by the induction of xenotolerance (Dorling & Lechler, Xenotransplantation (1998) 5:234-45; Wekerle & Sykes, Annu Rev Med. (2001) 52:353-370).

Protocols have been developed to address these needs, specifically through the use of mixed chimerism and surrogate telerogenesis. The term mixed chimerism refers to the coexistence of donor and recipient hematopoietic cells, with donor representation that can be detected by non-PCR-based techniques; the state of mixed chimerism can also be referred to as macrochimerism. The chimeric immune system recognizes donor antigen as self, yet is capable of mounting a normal response to third party antigens. Although the end result is the same, allotransplantation, there are numerous methods for achieving mixed chimerism. However, the basic result is to mix donor and recipient hematopoietic cells to produce an allogeneic immune system. The benefits of mixed chimerism has been discussed in much detail and is readily recognized in the art (see, e.g., Gammie & Pham Curr. Opin. Cardiol (1999) 14(2):126-32; Wekerle & Sykes, Annu Rev Med. (2001) 52:353-370). Regardless of the advances made by this protocol, the basic problem of immunoreactivity remains as mixed chimerism does not provide a truly compatible immune systems.

To address this concern, a different protocol has been developed, one that is synergistic. Surrogate telerogenesis is a method for culturing human hematopoietic stem cells in a fetal animal (see, e.g., U.S. Pat. No. 6,060,049, and Beschorner et al., Trans. Proc. (2000) 32:994-995). Surrogate telerogenesis is based on the principle that immunological tolerance can be induced in fetuses. Once the cells are made tolerant to both the donor (human) and the recipient (animal), the cells are returned to the donor for reconstitution. Although addressing the shortcomings of allotransplantation, surrogate telerogenesis requires a rapid induction of tolerance and proliferation of the human stem cell in a xenohost. Often, this requires high levels of human stem cells, which may not be available, especially if the individual has immunological problems, such as autoimmune diseases, AIDS, cancer, etc.

In view of the expanded approach to treatment of many severe diseases associated with bone marrow transplantation (also referred as hematopoeitic/stem cell transplantation), a method for achieving high rates of engraftment of bone marrow cells from HLA-nonmatched donors, with low incidences of graft rejection and GVHD, would be highly desirable. The reliable induction of a robust, drug-free, permanent state of immunological tolerance could provide a solution to these pressing problems in the field of transplantation. Thus, strategies for the induction of transplantation tolerance have the potential to dramatically improve the prospects for graft recipients and open the door to a whole new era of transplantation using xenografts.

### SUMMARY OF THE INVENTION

In a general embodiment of the present invention, there are provided transplantable immune systems from non-human animals, and the cells, tissues and organs from the animal, which are tolerant to antigens taken from an individual human, or from multiple humans. In a more particular aspect of the present invention, the non-human animals are generated by the presentation of antigens from a human (i.e., the intended human recipient of the immune system) into an immunodeficient animal, such as a neonatal or fetal animal; and thereafter reconstituting the immune system of a recipient (i.e., a human recipient after being made immunodeficient) with the tolerized immune system harvested from the non-human animal. Subsequent to reconstitution of the immune system, the human recipient can continue to receive any other organs, tissue or cells derived from the non-human animal donor. The present invention also provides methods for generating the tolerized animals and the organs, tissues and cells thereof, as well as methods for the use of the organs, tissues and cells of the tolerized animal.

In an alternative embodiment of the present invention, the non-human animal donor can be tolerized with antigens from multiple humans so as to not be specific for one individual. This allows the animal to be used as both a universal donor for the group of individuals, or alternatively allows the animal to be the donor of the immune system but allows the other human individuals or other animals to be donors of other tissue.

In a preferred embodiment, multiple animals are infused with antigens from the intended immune system graft recipient. The best animal is selected on the basis of the degree of immune tolerance conferred by the antigens and the best animal is then used as a source of tolerant cells and factors and organ graft. Multiple tolerized animals also generate several sources for cell, tissue or organ transplantation, which can be harvested at from animals at different developmental stages, i.e., immature non-fully differentiated or mature differentiated cells, tissues or organs from fetal, juvenile and/or adult animals.

In a more particular aspect of the present invention, the method for generating the tolerant or tolerized immune system comprises multiple steps, primarily two. The first step involves generating the immune system by presenting, at least, the important transplantation antigens (including major histocompatibility antigens or MHC, minor histocompatibility antigens, arid tissue-specific antigens) of an individual human patient into an immunodeficient animal that can develop immune competence Such animals include immunologically immature non-human mammals, preferably neonatal or fetal. Any mammal is contemplated for use in the present invention, such as primates and non-primates.

The sources of these various antigens include, but are not limited to, human progenitor and stem cells, immature cells, mature cells and tissues, and products derived from the cells or tissues of the recipient. It may be necessary to process the antigens prior to exposure to the immunologically immature animal. Processing may include purification, characterization, and the removal of pathogens.

Several different methods can be used to expose the immunologically immature animal to the human antigens. Examples of such methods include, but are not limited to, intravenous or intra peritoneal injection, surgical introduction, intrauterine introduction, and introduction by techniques commonly used in the fields of molecular biology and genetic engineering including, but not limited to, vectors, viral vectors, transgenic methods, and the production of chimeric animals.

Once the animal has been tolerized, the immune system of the animal can be harvested and enriched/purified for cells that can reconstitute the recipient immune system. Sources of animal donor immune system cells and tissues may include hematopoietic and lymphoid cells, including lymphocyte progenitors and stem cells derived from bone marrow or peripheral blood, thymus; and lymphoid tissue such as is found in lymph glands, dendritic cells, macrophages, lymphocytes and plasma cells and endothelial cells. The cells can be modified outside of the intended organ graft recipient prior to reconstitution.

The cultured tolerized cells can then reconstitute the intended immune system graft recipient. Graft vs. host disease (GVHD) is minimized because of the induction of tolerance to the human antigens prior to the transplant. Additional precautions can be added to decrease the likelihood of GVHD, including modifying the cells prior to reconstitution. In addition, the human recipient is depleted of his own immune system to minimize or avoid subsequent host vs. graft disease, or graft rejection.

Once tolerance to human antigens in the non-human animal is achieved, and the non-human immune system is immunologically mature (i.e., immunocompetent but tolerant of the recipients and animals antigens), the human is prepared to be the recipient of an immune system transplant. Transplantation of the immune system, and pre-transplantation methods is performed according to established clinical practices. Current clinical transplantation practices may be altered and optimized to exercise the advantages offered by the non-human donor marrow described in this invention.

The final result is a human with a xenogeneically derived functioning immune system that recognizes human tissues as “self”. In addition, the transplanted immune system will continue to recognize, as “self”, tissues derived from the animal (which may be a member of an inbred, or cloned, genetically homogeneous strain) that donated the immune system. Such tissue includes grafts, cells, proteins and molecules, that are less susceptible to rejection by the recipient as they are tolerized to the antigens of the recipient and the animal.

The process of immunological reconstitution, as described above, could be repeated multiple times in order to sustain a functioning immune system in the human recipient. Therefore a limited period of survival of the xenogeneic marrow transplant in the human would not represent a major obstacle to the success of this invention. Furthermore, by using a genetically homogeneous inbred or cloned species as immune system donors, and by inducing tolerance to the human patient in multiple animals at the same time, additional marrow transplants (subsequent to the initial xenogeneic reconstitution) could be performed without the need for additional preparation of the human recipient.

In summary, this invention describes a general procedure that would allow humans to receive xenogeneic immune system transplantations without the occurrence of graft vs. host disease or graft rejection. This invention has far reaching medical benefits in the treatment of AIDS, cancer therapy, organ transplantation and other areas.

Additional objects and advantages of the invention are set forth in part in the description which follows, and in part are apparent to one skilled in the art from the description. The objects and advantages of the invention also may be realized and attained by means of the instrumentalities and combinations particularly pointed out in the appended claims.

### BRIEF DESCRIPTION OF THE DRAWINGS

FIG. 1 illustrates one embodiment of the invention by means of a flow chart showing the tolerization of the animal, in this case a pig, and then reconstitution of the immune system of the human with the tolerized immune system of the pig.

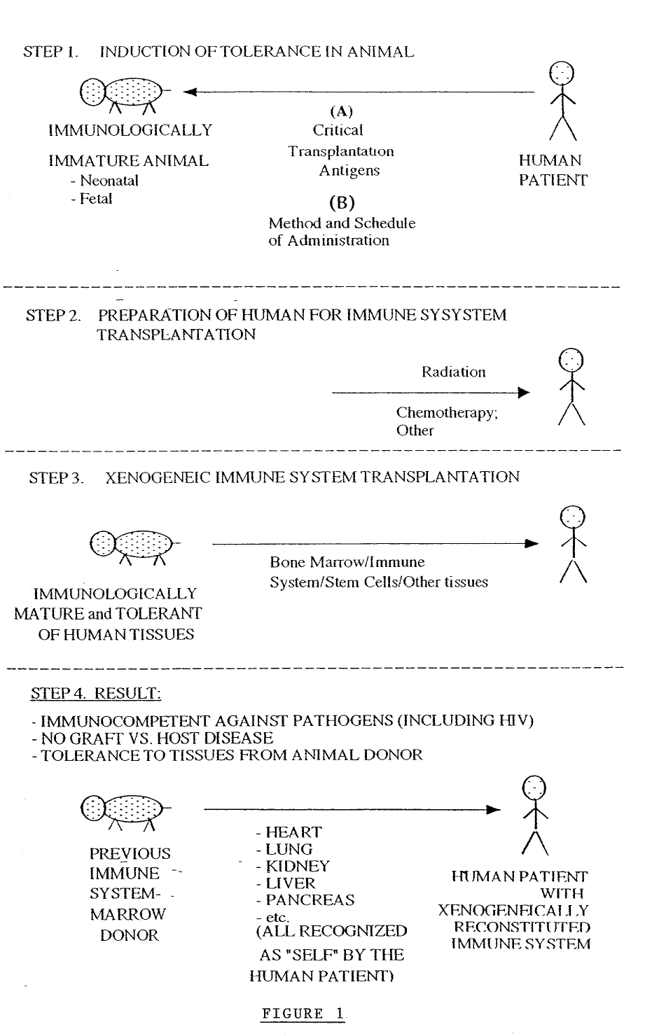

### DETAILED DESCRIPTION OF THE PREFERRED EMBODIMENT

In accordance with one aspect of the present invention, there are provided immune tolerant non-human animals wherein the animals comprise a non-human immune system tolerized to human molecules, cells or tissues, preferably molecules, cells or tissues derived from a specific human designated to be the recipient of the immune system. As described herein, the term “immune tolerant non-human animals” refers to animals, preferably mammals, having an immune system which recognize particular foreign molecules, cells, or tissues (including organs) as “self”, but which react normally with third party unrelated antigens (i.e., “foreign”). As described herein, the term “tolerant” (or variations thereof) refers to the acceptance of an immune system, and the components thereof (e.g., molecules, cells, tissues or organs) to particular antigens as “self”. Thus, the term “tolerized” is defined as the induction of tolerance of an immune system to the molecules, cells or tissues of a particular human recipient presented to the immune system. Tolerant immune systems are unresponsive or demonstrate a decreased immune response to particular molecules, cells or tissues similar or identical to molecules, cells or tissues (antigenically identical or similar substances) used to induce tolerance, but are immune competent in all other aspects (i.e., to antigenically distinct substances).

As readily recognized by those of skill in the art, the immune system refers to the complex network of specialized cells and organs which defends the body against attacks by “foreign” invaders. Further discussion regarding the immune system, and the organs and cells comprising the immune system, and the development thereof can be found in Abbas et al., Cellular and Molecular Immunology 4th edition (W B Saunders Co., 2000) 553 pages, Goldsby et al., Kuby Immunology 4th edition (W H Freeman & Co., 2000) 670 pages; Tizard IR, Veterinary Immunology: An Introduction 6th edition (W B Saunders Co., 2000) 482 pages; each hereby incorporated by reference.

Antigens are defined in Rosen F. S., et al., eds., Dictionary of Immunology, 1989, Macmillan Press, UK, p. 13, as “substances that can elicit an immune response and that can react specifically with the corresponding antibodies or T cell receptors. An antigen may contain many antigenic determinants.” Antigenically identical substances, as discussed herein, contain the same antigenic determinants and are reactive with the same antibodies and T cell receptors. Antigenically similar substances, as discussed herein, share many of the antigenic determinants and react with many of the same antibodies and T cell receptors. Antigenically distinct substances, as discussed herein, share few, if any, antigenic determinants and react with different antibodies and T cell receptors.

Immune response, as discussed herein, includes acquired immune responses that involve the proliferation of T and/or B lymphocytes specific to the inducing antigen.

In contrast to immune tolerance, immune competence, as discussed herein, is defined as the ability to mount a normal immune response to antigenically distinct molecules, cells or tissues, but immune competent animals may exhibit decreased response to molecules, cells, or tissues antigenically similar or identical to the animal. An example of immune competence would be the ability to promptly reject a skin graft from an allogeneic donor (typically in 6 to 12 days) but accept a synergistic or autologous graft indefinitely.

Immune deficiency, as discussed here, refers to an impairment of an animal's (including the tolerized animals and human recipients) immune system to react to antigenic moieties such as molecules, cells or tissues, as compared to immune reactions of a normal mature animal. An example of immune deficiency would be an animal that accepts a new skin graft from an unrelated donor for a prolonged period, as compared to immediate or near-immediate rejection in a normal host. Immune deficiency is distinct from immune tolerance in that immune deficient animals will be unresponsive to most, if not all, antigenic substances; distinct, similar or identical. Within the current context, examples of immune deficient animals would include immature animals, including neonatal or fetal animals, animals after total body lethal irradiation, animals engineered to be immune deficient, and the like. Neonatal or fetal non-human animals, depending on species, are immunologically unresponsive to (certain) antigenically distinct substances, including substances derived from xenospecies such as humans, because the immune system is immature and/or does not detect the substance as lethal or dangerous. Lethally irradiated animals are immune deficient and unable to reject antigens because the immune system was destroyed or functionally impaired by the irradiation. Animals engineered to be immune deficient include animals with SCID.

An organ graft is herein defined to mean a solid organ, a non-solid or partially solid tissue to be transplanted. Solid organs include organs comprising the gastrointestinal, cardiopulmonary, neural, sensory, reproductive, and the like systems. Non-solid and partially solid organs include stem cells, mature cells and immature cells, and the like.

An organ graft recipient is defined herein to mean an animal such as a human intended to be the final recipient of an organ graft.

A wide variety of positive and negative, central and peripheral mechanisms has evolved to regulate the immune response, including suppression, negative and positive selection such as clonal deletion and clonal inactivation, cytokine-dependent immune deviation, energy, (See, e.g., van Parijs L et al., Novartis Found Symp (1998) 215:5-20, 33-40), and the like. Each operates to varying degrees in the generation and maintenance of tolerance, although their relative contribution may vary depending on the nature of the antigen and the location in which “tolerization” occurs, i.e., central or peripheral (see, e.g., Roitt et al., Immunology 5th edition (Mosby, 1998), Bluestone et al., J Am Soc Nephrol (2000)). Thus, in every response, whether positive or negative, the factors mobilized and the balance between protection and damage depend upon the quality, quantity, location, and timing of immunogen presentation, as well as upon properties of the host. (See, e.g., Silverstein & Rose, Semin Immunol (2000)12(3):173-8; discussion 257-344; Butler et al. Plast Reconstr Surg. (2000) 105(7):2424-30; discussion 2431-2; Min B et al., Int Rev Immunol. 2000;19(2-3):247-64; Garza KM et al., J Immunol. 2000 Apr 15;164(8):3982-9; Auchincloss, H. Jr., Transplantation (1988) 46(1):1-20; each incorporated herein by reference).

The normal immune system is capable of specifically differentiating between “self”/“benign” (referred herein as “self”) and foreign/toxic (referred herein as “foreign”) entities , with foreign/toxic entities including infectious agents. The ability to differentiate self from foreign entities is established naturally throughout an animals life, especially during fetal development, when the developing immune system of the fetus is programmed to recognize presented antigens as self; i.e. as antigens of the fetus.

Immunological tolerance to particular substances (e.g., molecules, cells or tissue) that are normally antigenic can be induced in an animal by exposing the immune system of the animal to identical or similar substances. Although shown in adults, immunological tolerance has been observed mostly in animals wherein the immune system is still immature (during fetal development or during the neonatal period, depending upon the species and the antigens). (See, e.g., Traub, E. J. Exp Med. 19??) 68:229-50; Nossal G J, Annu Rev Immunol. (1983) 1:33-62; Nicoletti et al., Mol Med. (2000) 6(4):283-90; Kaplan et al., Semin Thromb Hemost. (2000) 26(2):173-8; Bluestone et al., J Am Soc Nephrol (2000) 11:2141-2146; Grable & Karin, Int. Immun. (1999) 11(6):907-913; each herein incorporated by reference). The animal becomes tolerized to the infused substances and to antigenically similar substances, but immunocompetent with respect to any other distinct antigens.

Accordingly, in one embodiment of the present invention, there are provided methods for producing immune tolerant non-human animals, and the immune cells and tissues thereof, by tolerizing the animal to antigens (i.e., molecules, cells and tissues) from a particular individual, e.g., a human designated to be the recipient of the tolerized immune system. More specifically, the method comprises the steps of obtaining a plurality of antigens (e.g., molecules, cells or tissues) from a particular recipient, and presenting these antigens to an immune deficient non-human animal (inducing tolerance in the animal to the recipient's molecules, cells or tissues). The immune system, and components thereof, of the non-human animal are thereby programmed to be specifically tolerant to antigenically similar or identical substances as the molecules, cells and tissues originally presented into the animal. Thus, for example, an immunologically immature non-human mammal is infused with antigenic substances (such as molecules, cells or tissue from a human subject in need of a new immune system), and thereafter, allowed to develop into an immune competent animal tolerant to antigenically identical or similar substances as the presented substances. Once fully immunologically competent, the immune cells/tissue can be taken from the developed animal and reconstituted or regrafted into the particular recipient, as described herein.

The animal can be presented with a myriad of antigens similar or identical to the antigens desirably recognized as self. Several classes of antigens can be presented individually or simultaneously, including major histocompatibility antigens (MHC), minor histocompatibility antigens, and tissue-specific antigens, to produce maximum tolerance. Preferably, a variety of antigens will be introduced into the animal as the use of MHC antigens alone will not likely be sufficient to produce clinically useful tolerance. The sources of these various antigens include, but are not limited to, human stem, immature and mature cells and tissues, and products derived from the aforementioned cells or tissues.

In addition, to ensure that tolerization occurs, the non-human animal can be challenged multiple times by presentation of antigens from the recipient throughout the life of the animal. Challenging the animal at several stages throughout the life of animal ensures that the animal will be immune tolerant to the antigens of the recipient, and also provide a method for culling those animals which evoke an immune response thereto. Those of skill will recognize the most suitable method for tolerizing animals based on the animal and the antigen.

In a preferred embodiment of the present invention, the non-human animal is tolerized with antigens (molecules, cells or tissues) from a recipient having abnormal cells, tissues and/or organs. More preferably, the animal is tolerized by presentation into the animal antigens identical or similar to the abnormal cells, tissues or organs. In general, and further described herein, the term “abnormal” refers to cells, tissues, or organs derived from a human recipient which are functionally abnormal, e.g., diseased, infected or injured. Alternatively, it may not be desirable to tolerize the animal to the abnormal molecules, cells, tissues or organs, e.g., with respect to cancerous cells or virally infected cells. Instead, it may be desirable and/or necessary to process the antigens prior to exposure to the immunologically immature animal to remove the abnormal molecules, cells, tissues and/organs. Processing may include purification, characterization, and the removal of pathogens. The pathogens may be from the recipient, or they may be from the xeno-animal (xenozoonoses). To prevent xenozoonoses, screening and breeding practices known in the art can be employed to reduce the transmission of these pathogens.

Several different methods can be used to expose the immunologically immature animal to the human antigens. Examples of such methods include [[OTHERS]], but are not limited to, intravenous or intra peritoneal injection, surgical introduction, intrauterine introduction, and introduction by techniques commonly used in the fields of molecular biology and genetic engineering: including, but not limited to, vectors, viral vectors, transgenic methods, and the production of chimeric animals [see, e.g., Zhao et al., Transplantation (2000) 69(7):1447-51; Alberts, B., et al., Molecular Biology of the Cell, Third ed., (1995) Garland Publishing; Janeway, C. et al., Immunobiology, Fourth ed., (1999), Garland Publishing; Lodish, H. et al., Molecular Cell Biology, Fourth ed., (2000) H. H. Freeman Company; each hereby incorporated by reference] The intended use of the tolerized immune system will influence the mode of presenting the antigenic substances derived from the recipient. For example, intrauterine infusion would be useful for the generation of tolerized immune system (e.g., hematopoietic immature cells or other immature cells (progenitor and stem cells) which can be harvested from (multiple) the fetus or newborn for transplantation. In contrast, for solid organ transplantations (heart, kidney, livers, lungs, etc.), it would be more practical to induce tolerance by infusing tissue derived from the desired organ into the central and peripheral immune system .

Non-human animals contemplated for use in the invention method include any non-human species which have immune systems similar to human immune systems, particularly the immune system of the recipient. Many animals can potentially be used in the present invention, with each species offering advantages for select uses. Those of skill can readily select an animal for use in the present invention based primarily on the recipient and their needs: including the intended use (e.g., cell, tissue or organ transplantation), concordance and compatibility of the immune system and/or organ, gestation period (timeliness of invention method), size of the animal, ease or difficulty of cloning and/or genetic manipulation, and the like. The preferred non-human animals include vertebrates, specifically to all members of the class Mammalia except humans. Primates, artiodactyls, carnivores, rodents, and lagamorphs are particularly suitable for use in the present invention. The principles for tolerizing an animal (the immune system) with particular foreign molecules has been widely observed in the various animal species, particularly in cows, sheep, pigs, monkeys, mice, rats, and chickens (See e.g., Grabie & Karin, Int. Immun. Supra; Zanjani et al., Stem Cells (1997) 15 Suppl 1:79-92; Zanjani, et al., J. Clin. Invest. (1992) 89:1178-88; Duncan, et al., Transplant Proc. (1991) 23:841-3; Hasek, Cesk Biol. (1953) 2:265-70, 1953, each incorporated herein by reference). Those skilled in the art will readily recognize the available parameters which can be employed with respect to each animal. For example, the fetal period for developing immune tolerance can be readily established employing the methods described in these papers.

The primates, particularly the higher primates other than human, are the most suitable animals to tolerize from the standpoint of compatibility. Amino acid sequencing of proteins typically demonstrate greater than 90% homology with humans. Organs such as livers and hearts function well when transplanted into humans. In addition, the immune system of primates are concordant with humans, i.e., human recipients do not typically have preformed antibodies to the tissues of the primates. If the period for inducing tolerization is crucial, however, the gestation periods for primates (or each species) should be considered. While some of the lower primates, such as lemurs, have short gestation periods (132-134 days), the higher primates (chimpanzees, gorillas) have gestation periods approximating that of humans (267 days) that would

The artiodactyls, even toed ungulates, include several domesticated animals such as pigs, sheep, goats, and cows. Organs or proteins from several members have been demonstrated to be functional and useful in humans or have been proposed for transplantation. For example, porcine and bovine insulin, pig skin, sheep hearts, etc. have been used or proposed for therapeutic use.

The gestation periods vary between the members of this order. Pigs have a gestation period of 114 days. Sheep have a gestation period of 145 days. Cows have a gestation period of 282 days. Cows offer some unique features that are potentially useful for the present invention. The placental blood of all of the litter mates is shared, allowing infusion of one single calf to lead to tolerance to all of the litter mates. Because of their large size, cattle can provide more pancreatic islets than other animals for transplantation into diabetics. The limited numbers of pancreatic islets harvested from a human pancreas has been a major factor limiting the use of human allogenic transplantation of islet cells.

The carnivores, including dogs, cats, etc., have several features that are potentially advantageous. Many have short gestation periods (cats about 65 days, dogs about 63 days) and the newborn are relatively well developed. The canine and feline immune systems are very similar to the human immune system. For example, the feline immunodeficiency virus model in cats is one of the few animal models available for the study of AIDS. Following bone marrow transplantation, suppressor cells have also been identified in dogs.

In addition, cats and dogs have been commonly used as large animal models for transplantation, including bone marrow, lung, intestine, and bone transplants (Ladiges, et al., LAB. ANIM. SCI., 40:11-15, 1990; Henry, et al., AM. J. VET. RES., 46:1714-20, 1985). Human islets of Langerhans and hepatocytes have been shown to function well in dogs (Calafiore, ASAIOJ, 38:34-7, 1992; Petruzzo, et al., TRANSPL. INT., 4:200-4, 1991; Sussman, et al., HEPATOLOGY, 16:60-65, 1992). It may be anticipated therefore that canine islets and hepatocytes would function similarly in human recipients.

The rodents, including rats, mice etc., are potentially useful in the present invention as immune system donors because of their short gestation periods and rapid growth to maturity. For example, rats have a gestation period of only 21 days and grow to maturity in only 6 weeks. Because the immune system of rodents is very immature at birth, injecting rodents can induce tolerance within 24 hours of birth rather than by intrauterine injections.

Because of the short gestation and maturation periods, rodents are particularly useful for generating new strains and transgenic animals. In addition, because extensive research has been performed on rodents, those of skill in the art could readily generate rodent donors for harvesting of their immune systems and other tissues for therapeutic purposes. For example, the SCID mouse could be employed to generate lymphocytes that could be harvested into human recipients. In addition, using transgenic mice that produce human insulin or human growth factor, lymphocytes that are tolerant to the recipient could be produced within a few weeks by infusing the recipients antigens into a large number of newborn mice.

The lagomorphs, which include rabbits and hares, share with the rodents a very short gestation period and short maturation periods. Thus, they would also be useful for the development of new strains, including transgenic strains favorable for maturation of tolerized lymphocytes and providing functional organs or tissues. Their larger size would make these animals better candidates than rodents.

The ideal species should be phylogenetically close to the intended recipient of at least the immune system of the selected species. If organ graft is necessary, the physiology of the intended graft should be similar to the physiology of the recipient's organ or tissue to be replaced by the graft. Preferably, the organ graft recipient will be concordant with the animal; i.e. the organ graft recipient should not have natural antibodies to the animal. With the above criteria, the most optimal nonhuman animals for providing organs and tissues for human transplants are the non-human primates. Non-concordant animals being suitable for providing organs and tissues for human transplants include pigs, sheep, cows, dogs, horses, goats, etc.

Additional considerations influence the choice of species. For organ transplantation, the preferred transplanted graft is to be approximately the same size as the corresponding graft within the organ graft recipient. If suitable grafts to humans are required as soon as possible, the desirable traits would include a relatively short gestation period, a rapid growth after birth, and tolerance would be induced within the fetus. Consequently, with the additional considerations described above, pigs are preferable over primates because pigs have a gestation period of only 114 days and typically grow to over 59 kg by four months of age. However, if compatibly developed organs or tissues are necessary, then non-human primates are superior to pigs.

Although genetic engineering is not required, genetic modifications of the animals could significantly enhance and/or simplify the procedures, especially with respect to cloned animals (See, e.g., Campbell et al., Nature (1996) 7;380(6569):64-6 and Trounson & Pera, Reprod Fertil Dev (1998) 10(1):121-5). Genetic engineering of large mammals is commonly performed, including genetic modifications of sheep, cows, and pigs. Using techniques that are well known to those familiar with genetic engineering, potential genetic modifications could be made that complement the current invention. For example, potential genetic modifications could complement or facilitate the transplantation of the immune system, or alternatively, modify the function of the transplanted organ to better address the recipient's disease process.

For example, human decay activating factor (DAF) has been produced by a herd of transfected pigs. The insertion of human DAF into the ova of pigs produces a herd of animals more resistant to preformed antibodies. This would reduce the destruction of the organ xenograft caused by the binding of natural antibodies and activation of human complement.

Whereas discordant animals produce alpha galactosyltransferase (AGT) responsible for the development of oligosaccharides on discordant animal cells, humans, apes and old world monkeys fail to produce significant amounts of this enzyme. This failure is believed to be due to a mutation in the DNA responsible for AGT (Galili, Springer Semin. Immunpathol., (1993) 15:155-71). A strain of animals such as pigs containing a nonfunctional AGT may be produced using homozygous recombination to insert non-functional code into the pig gene for AGT or the corresponding promoter gene (Watson, et al., “Recombinant DNA,” Scientific American Books, N.Y., 1992, pp. 255-72). This alteration in the animal's cells would be better than administering complement inhibitors to the graft recipient, since the graft recipient's immune system could still interact with infected cells in the organ and protect it. By using genetically modified pigs or other animals with complement inhibiting factors as the animals, the need for plasmapheresis, ex vivo perfusion, or complement inhibiting drugs such as cobra venom factor could be significantly reduced.

The transplantation of xenografts would also justify the genetic modification of the animal or tissue (e.g., Yang et al., Biotechnol Annu Rev. (2000) 5:269-92). The modifications can lead to secretion of pharmacologically important human proteins, make the animal more resistant to infections, and enhance growth of the animals. For example, a strain of pigs producing increased amounts of alcohol dehydrogenase would be useful for liver transplants performed for alcoholic liver disease. Similarly, pigs producing an increased amount of human insulin in the pancreatic islets would be a useful source of tissue for transplantation treatment of either type I or type II diabetes mellitus. Pigs that produce increased amount of human erythropoietin would be useful for kidney transplants into patients with renal failure and anemia. By increasing the number of beta adrenergic receptors, heart xenografts could be produced that are stronger. Numerous other alterations that enhance the transplant organ for a particular disease will be apparent to the skilled worker.

In yet another preferred embodiment, the present invention contemplates tolerizing a plurality of animals, more preferably sibling animals before or after birth. Antigens from a human recipient can be presented via intrauterine injection to fetal sibling animals to create a line of animals tolerant to identical or similar antigens of the recipient. Thereafter, the best or most optimal animal can be selected based on tolerance of the animal's cells to the recipient's antigens. This will allow for selection of the most tolerant immune system, as well as sources for cell, tissue or organ graft lines.

In yet a further component of the present invention, the invention method comprises monitoring the amount or level of tolerance within the animal to recipients antigens. Following fetal culture or bone marrow transplantation, the surrogates are monitored to establish tolerance of the animals immune system to the antigens presented to the animal. The assays used to monitor tolerization will be readily apparent to the skilled worker, including challenging the immune system, the cells and tissues thereof, with antigens from the recipient and detecting any immune response. This can be accomplished in vitro or in vivo.

In a further component of the present invention, the immune system of the tolerized non-human animal is harvested for transplantation into the human recipient, the immune system preferably the hematopoietic progenitor and stem cells. Sources of animal donor immune system cells and tissues may include progenitor and stem cells derived from bone marrow or peripheral blood, cord blood, serum, thymus, spleen and/or other lymphoid tissues such as is found in lymph glands. These tissues or cells are sterilely removed from the selected animal. As disclosed herein, specific reference to the individual components of the immune system such as reference to transfer of the bone marrow, and the progenitor and stem cells should be regarded as exemplary transplantable tissue/cells of the immune system.

A variety of protocols are known in the art for isolating the desired cells, such as hematopoietic stem cells from non-human animals. See, for example, the Wheeler U.S. Pat. No. 5,523,226; Emery et al. PCT publication WO 95/13363, Shpall et al., Annu. Rev. Med. (1997) 48:241-51 and Spangurde, G J, Annu. Rev. Med (1994) 45:93-104. Procedures for obtaining bone marrow which contain progenitor or stem cells are known by those skilled in the art and are described in a variety of medical textbooks. For example, bone marrow cells can be obtained from a source of bone marrow, including but not limited to, ilium (e.g. from the hip bone via the iliac crest), tibia, femor, spine, or other bone cavities. Other sources of stem cells include, but are not limited to, embryonic yolk sac, fetal liver, and fetal spleen. Peripheral stem cells can be obtained from a donor, for example, by standard phlebotomy or apheresis techniques. For convenience, the following embodiments of the invention are described for bone marrow cells, although it should be understood that peripheral stem cells may be used as equivalent to bone marrow cells.

For isolation of peripheral progenitor and stem cells, a continuous-flow blood cell separator can be employed, using machines such as the COBE-Spectra and the Fenwall CS-3000, which processes the blood for progenitor and stem cells, returning the majority of the blood to the donor.

For isolation of bone marrow, an appropriate solution can be used to flush the bone, e.g., a salt solution supplemented with fetal calf serum (FCS) or other naturally occurring factors, in conjunction with an acceptable buffer at low concentration, generally from about 5-25 mM. Convenient buffers include HEPES, phosphate buffers and lactate buffers. Otherwise bone marrow can be aspirated from the bone in accordance with conventional techniques. The bone marrow harvests are preferably maintained in anticoagulation media, such as media containing about 10,000 units preservative-free heparin and about 50 cc anticoagulant (ACD) per about 100 cc tissue culture media. About 450 cc of bone marrow harvest is preferably added to about 50 cc of this media to which another about 50 cc of ACD is added.

Fetal or neonatal blood are also sources for the tolerized cells used in the present invention. Fetal blood can be obtained by any method known in the art. For example, fetal blood can be taken from the fetal circulation at the placental root with the use of a needle guided by ultrasound (Daffos et al., (1985) Am. J. Obstet Gynecol 153:655-660; Daffos et al., (1983) Am. J. Obstet. Gynecol. 146:985), by placentocentesis (Valenti, C., (1973) Am. J. Obstet. Gynecol. 115:851; Cao et al., (1982) J. Med. Genet. 19:8 1), by fetoscopy (Rodeck, C. H., (1984) in Prenatal Diagnosis, Rodeck, C. H. and Nicolaides, K. H., eds., Royal College of Obstetricians and Gynaecologists, London), etc.

In one embodiment of the invention, neonatal pluripotent stem and progenitor cells can be obtained from umbilical cord blood and/or placental blood (See, e.g., Cohen SB et al., Bone Marrow Transplant. (1998) 22 Suppl 1:S22-5. The use of cord or placental blood as a source of progenitor and stem cells provides numerous advantages. Cord blood can be obtained easily and without trauma to the donor animal, if further tissue or organ harvesting is necessary.

Cell collections should be made under sterile conditions. Immediately upon collection, the neonatal or fetal blood should be mixed with an anticoagulent. Such an anticoagulant can be any known in the art, including but not limited to CPD (citratephosphate-dextrose), ACD (acid citrate-dextrose), Alsever's solution, De Gowin's Solution, Edglugate-Mg, Rous-Turner Solution, other glucose mixtures, heparin, ethyl biscoumacetate, etc. (See Hum, B. A. L., 1968, Storage of Blood, Academic Press, New York, pp. 26-160).

After harvesting the immune system from the non-human animal, the harvested immune system from the animal can be enriched for tolerized cells (referring also to tissues and organs of the immune system) including immature lymphocytes, immature T and B cells, progenitor or stem cells, hematopoietic cells, and antigen presenting cells (APC), i.e., cells (preferably enriched) which are designated for infusion into the human recipient in need thereof and regeneration or reconstitution of recipient's immune system. Before administration into the recipient, the harvested immune system maybe enriched for tolerized cells by challenging the harvested immune system, or a portion thereof, with antigens from the recipient. Thereafter, the harvested immune system can be enriched for immature or undifferentiated cells by selecting for cells that express progenitor and stem cell surface antigens such as Thy-1, CD34, Flt-3 ligand and c-kit, in combination with purification techniques such as immuno-magnetic bead purification, affinity chromatography and fluorescence activated cell sorting.

As used herein, the terms “purified” or “enriched” refer to a population of tolerized cells that is at least about 60%, preferably at least about 70%, more preferably at least about 80%, and most preferably at least about 90% pure, with respect to a total cell population.

Although unnecessary because the immune system designated for transplantation is tolerized, a preferred embodiment of the present invention comtemplates removing fully differentiated tissue and cells including removing mature T and B cells. Various known techniques can be employed to separate the cells by initially removing lineage committed cells. The use of separation techniques include, but are not limited to, those based on differences in physical (density gradient centrifugation and counter-flow centrifugal elutriation), cell surface (lectin and antibody affinity), and vital staining properties (mitochondria-binding dye rho 123 and DNA-binding dye Hoechst 33342). Procedures for separation can include, but are not limited to, magnetic separation, using antibodycoated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique. Techniques providing accurate separation include, but are not limited to, FACS, which can have varying degrees of sophistication, e.g., a plurality of color channels, low angle and obtuse light scattering detecting channels, impedance channels, etc. Concomitantly or subsequent to a gross separation, which provides for positive selection, a negative selection can be carried out, where antibodies to lineage-specific markers present on dedicated cells are employed. Alternatively, genetically engineered animals or cells can be employed. In addition, those of skill can negatively select for lineage markers for CD34, Thy-1 or c-kit; and select for low staining with rhodamine-123 to achieve high enrichment of animal hematopoietic progenitor and stem cells (Spangrude, G. J. Annu. Rev. Med. (1994) 45:93-104 and Shpall et al., Annu. Rev. Med. (1997) 48:241-51).

Monoclonal antibodies are particularly useful for identifying markers associated with particular cell lineages and/or stages of differentiation. Such antibodies include antibodies to lineage specific markers which allow for removal of most, if not all, mature cells, while being absent on stem cells. The antibodies can be attached to a solid support to allow for crude separation. The separation techniques employed should maximize the retention of viability of the fraction to be collected. Various techniques of different efficacy can be employed to obtain “relatively crude” separations. Such separations are where up to 10%, usually not more than about 5%, preferably not more than about 1%, of the total cells present not having the marker can remain with the cell population to be retained. The particular technique employed will depend upon efficiency of separation, associated cytotoxicity, ease and speed of performance, and necessity for sophisticated equipment and/or technical skill.

While it is believed that the particular order of separation is not critical to this invention, the order indicated is preferred. Preferably, cells are initially separated by a coarse separation, followed by a fine separation, with positive selection of a marker associated with stem cells and negative selection for markers associated with lineage committed cells.

In a preferred embodiment of the present invention, hematopoietic progenitor and stem cells can be selected on the basis of cell surface markers (e.g. CD34), allowing for enrichment of the desired cells and depletion of contaminating tumor cells. The collected cells are stored frozen in a suitable cryoprotectant (e.g. dimethyl sulfoxide, hydroxyethyl starch) until needed. To reduce the volume, the collected marrow is usually processed to separate plasma from the cellular components. Removal of plasma can also eliminate red cell incompatibilities in allogeneic transplantation. The cell fraction can be enriched for mononuclear cells using density gradient techniques or automated separation methods and depleted of T cells using various cytotoxic agents. Collected marrow cells are cryopreserved according to established procedures that include controlled-rate freezing and the use of cryoprotectants. Stem cells are thawed in a warm water bath immediately prior to use to minimize loss associated with thawing.

Prior to transplantation into the recipient host, the progenitor and stem cells may be stimulated with a number of different growth factors (preferably obtained from fetal tissues such as human fetal thymus) that can regulate cellular or tissue reconstitution by affecting cell proliferation, differentiation, adhesion, growth and gene expression. Such growth factors include those capable of stimulating the proliferation and/or differentiation of cells and hepatic progenitor and stem cells. For example, growth factors (e.g., epidermal growth factor (EGF), transforming growth factor (TGF) or hepatocyte growth factor/scatter factor (HGF/SF), granulocyte-macrophage colony-stimulating factor (GM-CSF) or granulocyte colony-stimulating factor (G-CSF)), IL1, IL3, IL6, IL7, growth hormone, interferons, insulin-like growth factors, and the like may be utilized to accelerate the period in which certain cell types are generated. Other factors include cell adhesion molecules, extra cellular matrix molecules and the like. The cells may be stimulated in vitro prior to transplantation into the recipient subject. Alternatively, the progenitor and stem cells may be stimulated in vivo by injecting the recipient with such growth factors following transplantation.

The present methods and compositions can also employ tolerized cells genetically engineered (preferably by transfection) to enable them to produce a wide range of functionally active biologically active proteins, including but not limited to growth factors, cytokines, hormones, inhibitors of cytokines, peptide growth and differentiation factors. Methods which are well known to those skilled in the art can be used to construct expression vectors containing a nucleic acid encoding the protein coding region of interest operatively linked to appropriate transcriptional/translational control signals. See, for example, the techniques described in Sambrook, et al., 1992, Molecular Cloning, A Laboratory Manual, Cold Spring Harbor Laboratory, N.Y., Ausebel et al., 1989, Current Protocols in Molecular Biology, Greene Publishing Associates & Wiley Interscience, N.Y., and Dunbar, C E., Annu. Rev. Med. (1996) 47:11-20.

The terms “transfection” or “transfected with” refers to the introduction of exogenous nucleic acid into a mammalian cell and encompass a variety of techniques useful for introduction of nucleic acids into mammalian cells including electroporation, calcium-phosphate co-precipitation, DEAE-dextran treatment, liposome-mediated gene transfer, microinjection and infection with viral vectors. Suitable methods for transfecting mammalian cells can be found in Sambrook et al. (Molecular Cloning: A Cold Spring Harbor Laboratory press (1989)) and other laboratory textbooks. For transfection of an exogenous gene and regulatory sequences into progenitor and stem cells, it is preferable that these nucleic acids be contained in a plasmid or vector containing sequences or elements well known in the art for preparing the nucleic acid prior to transfection. Such sequences include those that enable the nucleic acid to be replicated, such as a bacterial origin of replication. Suitable plasmid expression vectors include CDMS (Seed, B., Nature 329, 840 (1987)) and pMT2PC (Kaufman, et al., EMBO .1 6 187-195 (1987)). It may be desirable to select for the bone marrow cells which have incorporated the nucleic acid after the transfection. This can be performed, e.g., by transfecting a nucleic acid encoding a selectable marker into the bone marrow cells along with the nucleic acid(s) of interest. Preferred selectable markers include those which confer resistance to drugs such as G41 8, hygromycin and methotrexate. Selectable markers may be introduced on the same plasmid as the gene(s) of interest or may be introduced on a separate plasmid. Following selection of transfected cells using the appropriate selectable marker(s), expression of the exogenous gene can be confirmed by various methods including immunofluorescent staining of the cells and measure of a biological activity of the protein encoded by the exogenous gene.

The term exogenous nucleic acid is intended to include any gene or fragment thereof, or modification thereof which is introduced into a cell. An exogenous gene of the invention can encode a protein or a peptide. An exogenous gene of the invention can also be a nucleic acid that is transcribed into RNA, but does not encode a peptide. For example, an exogenous gene can be a nucleic acid which, upon transcription into an RNA molecule is an “antisense” strand of another nucleic acid in or out of the cell, such that upon expression of the exogenous gene and synthesis of antisense molecules, a function in the cell is modulated. In another embodiment of the invention, the antisense nucleic acid inhibits or reduces expression of another nucleic acid, such as an endogenous nucleic acid.

In another embodiment, the exogenous gene encodes a therapeutic protein useful for treating a disease or condition. The exogenous gene can encode a secreted protein, a membrane bound protein, or an intracellular protein. Preferred exogenous genes encode a therapeutic protein. A therapeutic protein can be a steroid hormone, a steroid hormone receptor, a growth factor, a cytokine, a morphogenic protein, a polypeptide hormone, a polypeptide chemotherapeutic agent, a signal transduction factor and an intermediate. Preferred morphogenic proteins include bone morphogenic proteins (BMPs). Other preferred exogenous genes include multidrug resistance genes and genes encoding calcitonin or collagen components. Expression of multidrug resistance genes, e.g., MDR1, in bone cells should provide host resistance to a variety of chemotherapeutic drugs.

Other methods can be combined with the methods disclosed herein to promote the acceptance of the animals immune system by the recipient. For example, tolerance to the immune cells and tissue can also be induced by inserting a nucleic acid which expresses a donor antigen, e.g., a donor MHC gene, into a cell of the animal, e.g., a hematopoietic stem cell, and introducing the genetically engineered cell into the recipient. For example, stem cells can be engineered to express a human MHC gene, e.g., a human class I or class II MHC gene, or both a class I and a class II gene. When inserted into an animal's stem cells, expression of the recipients MHC gene results in tolerance to subsequent exposure to recipients antigen, and can thus induce tolerance to tissue from the recipient. These methods, and other methods which can be combined with the methods disclosed herein, are discussed in Sachs, U.S. Ser. No. 08/126,122, filed Sept. 23, 1993, hereby incorporated by reference and in Sachs, U.S. Ser. No. 08/129,608, filed Sept. 29, 1993, hereby incorporated by reference.

The cells and tissues of the animal's immune system can be administered to the recipient in an effective amount to achieve its intended purpose, i.e., reconstitution or regrafting of the immune system of the recipient. More specifically, an effective amount means an amount sufficient to lead to the development of a new immune system and restoration of immune function in the recipient, while remaining tolerant to recipient's and the animal's antigens.

Determination of effective amounts is well within the capability of those skilled in the art. The minimum number of cells needed to achieve the purposes of the present invention will vary depending on the degree and extent of damage, timeliness for reconstitution of the immune system and the size, age and weight of the recipient, and the like. For example, pluripotent stem cells can be administered in an amount effective to reconstitute the immune system of the recipient, whereas fully differentiated cells may require a greater amount. Preferably, between 5×10⁸ and 5×10¹⁰ organ graft recipient cells/kg organ graft recipient weight are obtained following harvest and enrichment. The in vitro tests of immune tolerance described previously may be used to assess the obtained lymphocytes and factors.

In yet another embodiment, the bone marrow cells and/or enriched oval cells can be administered to the recipient in one or more physiologically acceptable carriers. Carriers for these cells may include, but are not limited to, solutions of phosphate buffered saline (PBS) containing a mixture of salts in physiologic concentrations. In addition, the cells may be associated with a matrix prior to administration into the recipient host.

In one aspect, the methods of the present invention provide a population of tolerized cells transfected ex vivo with an exogenous gene. The transfected tolerized cells can be administered to a subject. Exemplary methods of administering the stem cells to subjects, particularly human subjects, include injection or transplantation of the cells into target sites in the subjects. The cells produced by the methods of the invention can be inserted into a delivery device which facilitates introduction by, injection or transplantation, of the cells into the subjects. Such delivery devices include tubes, e.g., catheters, for injecting cells and fluids into the body of a recipient subject, infusion bags or like containers for intravenous administration of the tolerized cell/tissue composition to a patient. In a preferred embodiment, the tubes additionally have a needle, e.g., a syringe, through which the cells of the invention can be introduced into the subject at a desired location. The tolerized cells can be inserted into such a delivery device, e.g., a syringe, in different forms. For example, the cells can be suspended in a solution or embedded in a support matrix when contained in such a delivery device.

As used herein, the term “solution” includes a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is known in the art. The solution is preferably sterile and fluid to the extent that easy syringability exists. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating the tolerized cells as described herein in a pharmaceutically acceptable carrier or diluent and, as required, other ingredients enumerated above, followed by filtered sterilization.

In addition, tolerized cells may be attached in vitro to a natural or synthetic matrix that provides support for the transplanted cells prior to transplantation. The type of matrix that may be used in the practice of the invention is virtually limitlessness. The matrix will have all the features commonly associated with being “biocompatible”, in that it is in a form that does not produce an adverse, or allergic reaction when administered to the recipient host. Support matrices in which the tolerized cells can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. See e.g., U.S. Pat. No. 4,298,002 and U.S. Pat. No. 5,308,701. These matrices provide support and protection for the tolerized cells in vivo and are, therefore, the preferred form in which the tolerized cells are introduced into the recipient subjects.

Next, the immune system of the non-human animal that was treated in Step 1 is transplanted into the human. As described herein, sources of animal donor immune system cells and tissues may include stem cells derived from bone marrow or peripheral blood, thymus; and lymphoid tissue such as is found in lymph glands. Graft vs. host disease does not occur (or is minimal) because of the induction of tolerance to the human tissues & antigens prior to the transplant. This allows further procedures such as graft transplantation as graft rejection does not occur or is minimal.

In yet another component of the present invention, the human is prepared to be the recipient of an immune system transplant, according to established clinical practices (Janeway, C. et al., Immunobiology (Garland Publishing; 1999) pg. 435-440; Goldman & Bennett, Textbook of Medicine, (W. B. Saunders; 2000), pg. 987-991; each incorporated herein by reference. Current clinical transplantation practices may be altered and optimized to exercise the advantages offered by the non-human donor marrow described in this invention.

Accordingly, the invention features restoring or inducing immunocompetence (e.g., restoring or promoting the thymus-dependent ability for T cell progenitors to mature or develop into functional mature T cells) in the recipient, e.g., a human. The invention includes the steps of introducing into the recipient the harvested immune cells, e.g., xenogeneic thymic tissue, preferably fetal or neonatal tissue, so that animals immune cells can mature in the recipient.

An alternate approach includes performing bone marrow transplantation on the recipients. The recipients receive either lethal total body irradiation or high dose chemotherapy to destroy their immune system. The recipients immune system is treated to deplete the immunologically committed or potentially committed cells and/or tissue, i.e., hematopoietic stem cells, lymphocytes, T and B cells, and the like. The treated or enriched tolerized cells harvested from the animal are then infused into the recipient.

The graft recipient may require treatment before the adoptive transfer of the tolerized cells harvested from the animal, with the treatment including therapy (for example, chemotherapy or radiation) to allow for establishment of the animal's immune system cells and tissue into the recipient's immune system. For the cells to establish in the recipient, hematopoietic space may have to be created (preferably prior to thymic tissue or hematopoietic stem cell transplantation). In addition, if the animal and graft recipient are discordant; i.e. the recipient is serologically reactive to the animal (has natural antibodies against the animal's tissue, including the transplanted immune system), additional therapy is required to block a hyperacute rejection of the animal's tissue. The human may be depleted of his own immune system to create space or to minimize or avoid subsequent host vs. graft disease, or graft rejection, for example, by one or more of: by total lymphoid irradiation or total body irradiation, the administration of a immunosuppressant or myelosuppressive drug (as is described in U.S. Ser. No. 08/220,371), the administration of a hematopoietic stem cell inactivating or depleting antibody, and the like, to deplete the bone marrow of the recipient (preferably prior to thymic tissue transplantation). Plasmapheresis, splenectomy, cobra venom factor, and/or the use of soluble complement receptors may be used for the additional therapy. These additional therapy efforts are generally directed at circulating factors in the recipient at the time of transplant; the cells and factors transplanted into the recipient from the animal may prevent the similar development of these factors at a later period.

Other preferred embodiments include depleting or otherwise inactivating natural antibodies, e.g., by one or more of: the administration of a drug which depletes or inactivates natural antibodies, e.g., deoxyspergualin; the administration of an anti-IgM antibodies; or the absorption of natural antibodies from the host's blood, e.g., by contacting the host's blood with donor antigen, e.g., by hemoperfusion of a donor organ, e.g., a kidney or a liver, from the donor species. In other preferred embodiments the method includes: (preferably prior to or at the time of introducing the thymic tissue into the recipient) depleting, inactivating or inhibiting recipient natural killer (NK) cells, e.g., by introducing into the recipient an antibody capable of binding to NK cells of the recipient, to prevent NK mediated rejection of the thymic tissue; (preferably prior to or at the time of introducing the thymic tissue into the recipient) depleting, inactivating or inhibiting host T cell function, e.g., by introducing into the recipient an antibody capable of binding to T cells of the recipient (OKT3); (preferably prior to or at the time of introducing the thymic tissue into the recipient) depleting, inactivating or inhibiting host CD4⁻cell function, e.g., by introducing into the recipient an antibody capable of binding to CD4, or CD4⁺cells of the recipient. An anti-mature T cell antibody which lyses T cells as well as NK cells can be administered. Lysing T cells is advantageous for both thymic tissue and xenograft survival. Anti-T cell antibodies are present, along with anti-NK antibodies, in anti-thymocyte anti-serum. Repeated doses of anti-NK or anti-T cell antibody may be preferable. Monoclonal preparations can be used in the methods of the invention.

Methods of inducing tolerance, e.g., by the implantation of hematopoietic stem cells, disclosed in Sachs, Cosimi, and Sykes, U.S. Ser. No. 07/838,595, filed Feb. 19, 1992, hereby incorporated by reference, can also be combined with the methods disclosed herein.

Prior to the adoptive transfer of tolerized from the animal to the graft recipient, blood drawn from the graft recipient is then evaluated for tolerance against the animal's harvested immune system as described herein to avoid GVHD, e.g., using the in vitro methods described above. If the recipient's blood is reactive to the harvested and tolerized cells, additional steps may be necessary to delete the recipients immune system. Alternatively, it may indicate that the cells from the animal are not completely tolerized to the recipient. If on the other hand the cells are not reactive, the transfer of the animal's immune system is permissive. Following adoptive transfer, satisfactory tolerance of the animal's immune system into the recipient should also be tested allowing further treatment of the recipient, e.g., receipt of a surrogate organ or tissue using standard transplant procedures. Those of skill will know how to test for rejection, including using the methods described herein.

In yet a further component of the present invention, the tolerized immune system of the non-human animal, including the cells and tissues thereof, can be administered or transplanted to the recipient either locally or systematically. As used herein, the term “recipient” is intended to include human subjects in need of reconstitution, regraftment or regeneration of an immune system. It is believed that the invention procedure results in a permanent restoration of the hematopoietic system in most instances. However, with some disorders, repeated transplantations may be necessary.

Methods for carrying out bone marrow and peripheral blood stem cell transplants are known in the art. For a review, see Benz and McArthur, eds. Snyder et al., “Transfusion Medicine” in, Hematology 1994, American Society of Hematology, 96-106, 1994; Atkinson, K., Clinical Bone Marrow and Blood Stem Cell Transplantation; 2nd edition (Cambridge Univ Pr (Short), 2000) 1500 pages; Ball et al. (eds.) Hematopoietic Stem Cell Therapy (Churchill Livingstone, 2000) 800 pages; Donnall et al., Hematopoietic Cell Transplantation, 2nd edition (Blackwell Science Inc., 1999); each herein incorporated by reference.

For example, the tolerized cells are introduced to the recipient's circulatory system by a suitable method such as intravenous, subcutaneous, or intraperitoneal injection or infusion. Intravenous injection or infusion are the presently preferred methods. Generally, a composition will be prepared that comprises the tolerized tissue/cells and a physiological solution, such as saline, which is suitable for use as a vehicle for the administration of the tolerized tissue/cells to the circulatory system. The cells/tissue may first be rinsed in the solution to remove residual culture medium or, if the cells are freshly thawed, remove residual cryopreservation medium. If the tolerized tissue/cells have been frozen, it is preferable to thaw them, culture them in vitro in a growth medium (i.e. a culture medium containing growth factors that induce proliferation), and passage them at least once prior to transplantation. This ensures the viability of the cells and removes excess cryopreservant. The final concentration of tolerized tissue/cells is not critical, provided that a sufficient number of cells are administered for reconstitution of recipients immune system. For ease of administration and for the patient's comfort, it is usually preferred to minimize the total volume of cell suspension administered provided that the cells can be easily injected or infused into the patient without clumping. The final concentration will generally be in the range of about 10to 10precursor cells/ml.

Once suitable numbers of the invention cells/tissue needed for a particular purpose are obtained, they are transplanted into a patient using treatment regimes known to those skilled in the art for transplantation of hematopoietic stem cells. For the treatment of humans, much information is available in the art about techniques for the transplantation of hematopoietic stem cells for the treatment of various disorders (Bensinger et al. J. of. Clin. Oncology, 13(10):2547-2555 (1995); and Tricott et al., Blood 85(2):588-596). These references describe clinical trials for the transplantation of autologous peripheral blood stem cells for the reconstitution of a patient's hematopoietic system.

The process of immunological reconstitution, as described above, could be repeated multiple times in order to sustain a functioning immune system in the human recipient. Therefore a limited period of survival of the xenogeneic marrow transplant in the human would not represent a major obstacle to the success of this invention. Furthermore, by using a genetically homogeneous inbred or cloned species as immune system donors, and by inducing tolerance to the human patient in multiple animals at the same time, additional marrow transplants (subsequent to the initial xenogeneic reconstitution) could be performed without the need for additional preparation of the human recipient.

The principal goal of the present invention is the induction of antigen-specific tolerance, tolerization, in the immune system of a non-human animal, wherein tolerance is specific to the antigens of a particular recipient. Tolerization of immune deficient animals, such as fetuses, allows those of skill to generate immune competent animals tolerant to antigens from a particular antigen. The animals, in general terms, become incubators for transferable immune systems (cells and tissues therefrom) which can reconstitute or regraft the immune system of a particular recipient without the fear of immunogenic complications such as GVHD. Once established in the recipient, the reconstituted immune system allows, if necessary, the further transplantation of any cell, tissue, organ or system from the animal that has had its immune system deleted but now recognizes its reconstituted immune system as “self”and vice versa.

The use of such animals for developing immune tolerance provides the flexibility to perform procedures considered either impractical or unethical if applied to human recipients. Multiple animals may be tolerized, and the animal providing the best tolerance may then be selected for harvesting the tolerant cells and factors. Alternatively, or in addition, multiple tolerized animals tolerant to the recipient (and animal) generates several sources of cells, tissues or organs from available to the recipient.

The final result is a human with a xenogeneically derived functioning immune system that recognizes human tissues as “self”. In addition, the transplanted immune system will continue to recognize as “self”, tissues derived from the animal (which may be a member of an inbred, genetically homogeneous strain) that donated the marrow.

Hematopoietic transplants have been used to treat a variety of diseases including, but not limited to aplastic anemia, deficiencies of the immune system, autoimmune diseases, cancers affecting the hematopoietic system, such as lymphomas, leukemias, osteosarcomas, and the like, sickle cell disease, osteoporosis and others (see O'Reilly, R. J., Blood 62:941-964 (1983); Thomas, E. D. Blood Cells, 17:259-267 (1991); Marmont, A. M. Bone Marrow Transplant 11:3-10 (1993); Atkinson, K., Clinical Bone Marrow and Blood Stem Cell Transplantation supra; Ball et al., Hematopoietic Stem Cell Therapy supra; Donnall et al., Hematopoietic Cell Transplantation supra; each hereby incorporated by reference.). Transplantation of the invention tolerized cells/tissue of the animal's immune system can be used in place of bone marrow for treatment of these diseases. In addition, intravenous administration of tolerized cells/tissue into patients with autoimmune disorders, may alleviate the symptoms of the disorder. (see, Kenyon, N. S., IBC on Hematopoietic Stem Cells (1997)). The invention cells/tissues may also be altered by extrinsic or epigenetic means and implanted into normal or non-diseased individuals so as to endow them with a hematopoietic system with supra-normal functions.

In general, the clinical benefits of this invention would occur mainly in several areas of medicine and in the treatment of many disorders and diseases:

(1) Hereditary and Acquired Immunodeficiency Disorders, including AIDS

Because the human immunodeficiency viruses would not be able to infect the cells of the non-human immune system donor, an individual reconstituted with a xenogeneic immune system would be protected from the most devastating immunological effects of HIV infection. It is plausible to believe that a reconstituted individual would achieve a significant clinical remission from, or be cured of, AIDS.

It is plausible to expect that other hereditary and acquired immunodeficiency disorders [14] would be cured by transplanting, into the human, an immunocompetent xenogeneic immune system. Examples of other hereditary and acquired immunodeficiency disorders Include: ataxia telangiectasia, Bloom's syndrome; phagocyte deficiencies; complement deficiencies; Wiskott-Aldrich syndrome: DiGeorge syndrome; and immunoglobulin deficiencies. (14)

(2) Therapy of Cancers

(a) Presently, a factor that often limits the administration of radiation, chemotherapy and immuno-therapy to individuals with various malignancies is the development of bone marrow, or immune system, toxicity. Patients die from infections and hemorrhagic complications, secondary to marrow/immune depletion, before their malignancies can be cured. Xenogeneic reconstitution as described in this invention would alleviate deaths from marrow/immune depletion by providing an unlimited source of replacement marrow/immune tissues from the non-human animal donors. It is likely that malignancies now considered “incurable” could be cured, with presently available modalities, if these treatments could be given at much higher doses than are currently possible.

The improved reengraftment achieved using the methods of the invention is particularly useful in high-dose chemotherapy regimens. The hematologic toxicity observed with multiple cycles of high-dose chemotherapy is relieved by conjunctive administration of tolerized hematopoietic stem-cells. Diseases for which reinfusion of stem cells (cells not induced to be quiescent) has been described include acute leukemia, Hodgkin's and non-Hodgkin's lymphoma, neuroblastoma, testicular cancer, breast cancer, multiple myeloma, thalassemia, and sickle cell anemia (Cheson B. D., et al. (1989) Ann Intern Med. 30 110:51-65; Wheeler, C. et al. (1990) J. Clin. Oncol. 8:648-656; Takvorian, T. et al. (1987) N. Engl. J. Med. 316:1499-1505; Yeager, A. M. et al. (1986) N. Eng. J. Med. 315:141-147; Biron, P. et al. (1985) in Autologous Bone Marrow Transplantation: Proceedings of the First International Symposium, Dicke, K. A. et al., eds, p. 203; Peters, W. P. (1985) ABMT, supra, p. 189; Barlogie, B. (1993) Leukemia 7:1095; Sullivan, K. M. (1993) Leukemia 7:1098-1099). Treatment of such diseases can be improved by the method of the present invention of administering cells known to be quiescent and therefore capable of engrafting at an increased level in a host mammal which has or has not been subjected to myeloablation.

(b) In addition, the reconstituted xenogeneic immune system may be more effective, than the original human immune system was, at recognizing and eliminating neoplastic cells. To the degree that defective immune function or defective “immune surveillance” (15) contributed to the development of the malignancy, xenogeneic reconstitution may by itself contribute to a clinical remission.

(3) Leukemias Lymphomas and Related Hematological Malignancies

Bone marrow transplantation has been on efficacious therapeutic modality for these diseases for several years. but a limiting factor has been the availability of identical twins or other individuals with sufficiently matched transplantation antigens to act as marrow donors. With xenogeneic reconstitution and simultaneous induction of tolerance in multiple animals, animal strains (which could be inbred and genetically identical) would provide an essentially unlimited source of compatible donor marrow.

(4) Organ Transplantation

It will be possible to transplant organs (including heart, liver, kidney, lung, and pancreas) from the animal immune system-donor into the reconstituted human (see FIG. 2) because those organs will be recognized as “self” by the transplanted immune system (now hosted by the human). There are many diseases of primary organ dysfunction and failure, as well as many systemic illnesses that cause specific organ malfunction or failure. Prominent examples of specific organ malfunction or failure include heart dysfunction secondary to coronary artery disease or hypertension or cardiomyopathies; liver failure due to cirrhosis or hepatitis; lung failure due to emphysema or chronic bronchitis or cystic fibrosis or cancer; kidney failure due to hypertension or polycystic kidney disease, visual impairment in the aged due macular degeneration with degeneration of the retinal pigment epithelial cells, diabetes, and the like (see, in general, Ginns et al., Transplantation, 1st edition (Blackwell Science Inc., 1999) 942 pages; and Flye, M. W., Atlas of Organ Transplantation (W B Saunders Co., 1995) 376 pages; each hereby incorporated by reference). In general, transplantation as described herein will significantly reduce the incidence of rejection for a multiplicity of solid tissue organs, including skin, heart, kidney, liver, lung, intestines, pancreas, pancreatic islets, retina, cornea, bone, spleen, thymus, bone marrow, salivary glands, nerve tissue, adrenal glands, and muscle.

In addition, the present invention can also be used for facilitating transplant of organs that are fundamentally populations of cells transplanted as cell suspensions, such as bone marrow transplants (BMT), insulin-producing cells from islets of Langerhans of the pancreas, and the like (see, e.g., Weir et al., Ann Transplant. (1997) 2(3):63-8) The preimmune fetal environmental can develop stem cells, other than hematopoietic stem cells, such as neural stem cells, and the like. The fetal environment allows for proliferation of cell suspensions. By tolerizing multiple animals (cloned or sibling) to the same antigens, it is possible to provide sufficient cells for subsequent transplant and induce tolerance to these cells in a single procedure.

For example, pancreatic islets harvested from animal fetuses (10 to 14 weeks gestation) may be infused in a patient with type I diabetes mellitus, after the reconstitution of the animal immune system in the patient. Other examples include neural tissue for neurological diseases such as Parkinsons, Huntingtons, and the like.

The present invention further provides for generating animal lines tolerant to multiple recipients, human or animal. The non-human animal could be infused with antigens from multiple sources, becoming tolerant to both sources. For example, antigens from human siblings could be used to generate immune competent cells or tissues tolerant to both siblings. Once tolerance in the organ graft recipient is confirmed, the graft from the animal can be transplanted into the organ graft recipient. Alternatively, if the animal serves only as an incubator for the development of tolerance-inducing cells, then the graft from the prospective third party organ donor (sibling) is harvested and transplanted. Surgical transplantation techniques are well known in the art (see, e.g., Simmons, et al., “Transplantation,” in Schwartz, et al., 1989, eds. Principles of Surgery, McGraw-Hill, N.Y., pp. 387-458). The organ graft recipient is monitored for evidence of rejection of the organ graft in accordance with routine practice in the art, but the need for immunosuppressive therapy is significantly reduced compared to known methods of transplantation in the art.

If multiple tolerant animals are generated, one or more animals could be used to generate the immune system (which may be needed before hand in order to reconstitute the recipient's immune system), whereas the other animals, and the organs thereof, can be further developed. For example, if the animal has two or more of the graft organs, e.g. kidneys, then the original and best tolerant animal may be kept alive as a backup in the event of the first graft failing. Similarly, additional tolerized animals may be kept as backups for unique grafts; for example, grafts of hearts, or the additional tolerized animals may be kept in the event of failure of immune tolerance.

To provide universal tolerant cells, tissues and organs for emergency use, animals could be tolerized from multiple recipient members. For example, fetal pigs could be infused with antigens from multiple humans that express the most common histocompatibility antigens, a family. The resulting pig would then be expected to contain cells that would suppress the reaction of human lymphocytes sharing class I or II HLA antigens with the organ recipient against any other human antigens resident in the tolerized pig. The transplant organs from these tolerized pigs would also be expected to be (fully or partially) tolerant to antigens from any of the other recipient member. This would decrease the risk of rejection due to natural antibodies and cellular reactions to pig cells. This would be practical for many settings such as someone with fulminant hepatitis and liver failure or after a massive myocardial infarct when a transplant would be needed immediately.

(5) Hereditary and Acquired Bone Marrow Failure Syndromes

These syndromes include aplastic anemia; cytopenias; myelodysplasias; and myelofibrosis. Patients with these disorders would be expected to benefit, or be cured, from xenogeneic immunological reconstitution. In yet another embodiment, the invention provides methods for treating metabolic bone diseases, skeletal disorders or malignancies. Such skeletal disorders include osteoporosis (including post-menopausal osteoporosis), osteopenia (including drug-induced osteopenia), osteosarcoma, metastasis, and osteomalaciae. The invention also provides methods for treating osteosarcomas and other bone neoplasiae. The invention further provides methods for treating non-osseous tumors that metastasize to bone (e.g., breast cancer and prostate cancer). According to a preferred method of the invention, osteosarcomas and neoplasiae can be treated by selectively expressing a suicide gene in the malignant cells. The invention also provides methods for treating traumatic and iatrogenic bone lesions.

(6) Autoimmune Diseases

Autoimmune diseases that result from intrinsic abnormalities of the immune system are expected to benefit from xenogeneic reconstitution. Even autoimmune disease that results from the chronic, abnormal presentation of tissue antigens to a normally functioning immune system are expected to benefit from reconstitution with a “virgin” xenogeneic immune system. There are more than 500 diseases presently believed to have an autoimmune origin. See, e.g., Goldman & Bennett (eds), Cecil Textbook of Medicine (W. B. Saunders, 2000) pg. 1457-1462; Janeway et al., Immunobioloby (1999) pg. 490-509, 532-534). Such autoimmune diseases include, but are not limited to, type 1 insulin-dependent diabetes mellitus, pemphygus vulgaris, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biniary cirrhosis, pemphigus, pemphigoid, necrotizing vasculitis, myasthenia gravis, multiple sclerosis, systemic lupus erythematosus, Goodpasture's syndrome, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

Among the diseases that can be treated with success by stem cell transplantation are more than 20 otherwise fatal diseases that include the six or seven genetically different forms of SCID, various forms of congenital or genetically determined hematopoietic abnormalities, combinations of these two, certain anemias, osteopetrosis, a variety of high risk leukemias and several forms of severe life-threatening aplastic anemia. These diseases include SCID autosomal recessive with and without B cells (no ADA deficiency); SCID X-linked recessive without B cells; SCID autosomal recessive with ADA deficiency; Wiskott-Aldrich syndrome; Blackfan-Diamond syndrome; Fanconi anemia; severe neutrophil dysfunction; chronic granulomatous disease of childhood; severe (Kostman-type) agranulocytosis; immunodeficiency and neutropenia of cartilage-hair hypoplasia; infantile and late onset osteopetrosis; aplastic anemia-toxic chemical, idiopathic, immunological, and genetic (non-Fanconi); acute myeloid leukemia; chronic myeloid leukemia; Burkitt lymphoma, and recurrent acute lymphatic leukemia. Other diseases that have been treated recently with bone marrow transplantation include metabolic storage diseases such as Gaucher's disease, hemoglobinophaties such as thalassemia, and even some solid tumors such as neuroblastoma. In addition, BMT can be carried out before transplantation of an organ, e.g. kidney, from a same donor to a patient.

(7) Failures of Host Immunological Defenses Including Infections

Human immune defenses may fail to protect from invading pathogens. Infections with significant morbidity and mortality can result. Disorders in which patients suffer from serious disorders of host immune responses would be expected to benefit from xenogeneic immunological reconstitution. Examples of such disorders and infections include: leprosy; cytomegalovirus; herpes simplex; Epstein-Barr virus; and respiratory syncytial virus (Janeway et al., Immunobiology (1999), pgs 417-427, 455-456).

(8) Allergy and Hypersensitivity

Allergic and hypersensitivity reactions are common and can cause significant morbidity and mortality. Disorders in which patients suffer from serious allergic reactions would be expected to benefit from xenogeneic immunological reconstitution. Examples of allergic disorders include: asthma; drug allergies; food allergies; anaphylaxls; urticaria; eczema; and rhinitis (Janeway et al., Immunobiology supra pg. 461-488).

In accordance with the above teaching, this invention provides isolated organs for allogeneic or xenogeneic transplant either as a bridge or permanent transplant, where the animals are tolerized with antigens of the organ recipient and the immune system is preserved for subsequent transplant and optionally for transportation. Preservation of cells, tissues and organs for subsequent transplant is easily within the skill of the art.

The invention has thus far been described such that humans would be the recipients of xenogeneic immune system transplants. The methods in this invention could be used to allow other species to receive immune system transplants, and receive the benefits previously described for humans. Such applications may be desirable in the fields of animal husbandry, breeding, and in the protection of endangered species. In addition, the same methods described in this invention could be used to allow humans to be the recipients of immune system transplants from other humans. In such cases the important transplantation antigens from a human patient, or groups of patients, would be exposed to an immunologically immature human as a means of inducing tolerance to patient antigens, with subsequent harvesting of the exposed human immune system and other tissues for therapeutic purposes.

It will be apparent to those skilled in the art that various modifications may be made to the methods of surrogate tolerogenesis of the instant invention without departing from the scope or spirit of the invention, and these modifications and variations are within the contemplation of this invention provided they come within the scope of the appended claims and their equivalents.

This invention is further illustrated by the following examples which should not be construed as limiting. The contents of all references, patent, and published patent applications, including all drawings, figures, and tables cited throughout this application are hereby incorporated by reference.

### EXAMPLE

### Example 1

### Induction of Tolerance to Antigens derived from a Human Recipient

Endogenous Ag requirement for induction and maintenance of T cell tolerance has been extensively investigated in mice that express a transgenic Ag and/or its cognate transgenic TCR. In contrast, studies on tolerance for physiologically expressed self Ag and normal T cells are limited. Herein, we showed that the murine ovarian-specific ZP3 Ag is detectable from birth. Tolerance to ZP3 is detected in female relative to male mice. In comparison to males, 100-fold more ovarian peptide (pZP3) is required to elicit a comparable pathogenic response in females. Female tolerance to pZP3 was dependent on the presence of endogenous ovarian Ag, because neonatal ovariectomy converted the female response to that of males. Moreover, in female mice that were ovariectomized from the ages of 1-6 wk, the pZP3 responses were enhanced to the male level if ovaries were removed up to 7 days, but not 3 days, before adult challenge with pZP3. Thus, the physiologically expressed ZP3 Ag induces tolerance to pZP3, and the maintenance of tolerance is critically dependent on the continuous presence of the endogenous ovarian Ag. In contrast, exposure to endogenous ovarian Ag confined to the neonatal period is insufficient for the induction and maintenance of tolerance to ZP3.

### Example 2

### Fetal Tolerization

Vaccination procedure. Eight pregnant baboons with timed pregnancies are studied. Menstrual cycles are recorded three times per week for changes in the perianal sex skin (turgescence indicates follicular phase and deturgescence, the luteal phase). Ovulation occurs 2 d before deturgescence, and failure to mense approximately 14-17 d after deturgescence is the initial indicator of pregnancy. For the first experiment, the fetuses of three baboons are vaccinated at approximately 90, 120 and 150 days gestation with purified proteins isolated from human tissue by intramuscular injection. For the second experiment, the fetuses from five more baboons are similarly vaccinated. Four of the fetuses from the second experiment are given additional vaccinations as infants at 30 and 60 days after birth, to determine the effect of active immunization of the fetus on the ability of the neonate to respond to a similar vaccination. The vaccinations are scheduled so that the fetuses would be large enough to easily inject in utero, and the doses are given at intervals during the pregnancy such that development of a response during gestation could be detected. For fetal vaccinations, mothers are immobilized initially with ketamine (10 mg/kg) and xylazine (0.5 mg/kg), followed by sedation by anesthesia sufficient for surgery, with halothane (1.5%) and nitrous oxide (40%). In sterile conditions, a Teflon coated sonolucent 22-gauge needle is introduced through the anterior abdominal wall and uterus into the fetal thigh, using ultrasound guidance. Aspiration before injection is done to ensure that the needle is intramuscular, not intravenous or intraamniotic. All procedures are done with Institutional Animal Care and Utilization Committee approval and in accordance with the principles and procedures of the NIH Guidelines for Care and Use of Laboratory Animals.

Fetal blood sampling. We obtain fetal blood samples by percutaneous umbilical blood sampling at approximately 130 and 165 days of gestation. After sedating baboons by endotracheal anesthesia, we remove 2-3 ml of fetal blood using ultrasound guidance. Fetal heart rate is monitored intermittently during the procedure using Doppler ultrasound. Maternal EKG and blood pressure are also monitored during the procedure. Maternal blood is drawn from the cephalic vein just distal to the elbow simultaneously with each fetal blood sampling. To ensure that no maternal blood contaminated the fetal samples, an APT test (to detect adult hemoglobin) is done on all samples.

Radial immunodiffusion. IgM and IgG levels are initially determined by radial immunodiffusion using anti-human -chain- and anti-human -chain-specific reagents that cross-react with baboon IgM and IgG, respectively (The Binding Site, San Diego, Calif.). All mother-infant pairs are kept together in ‘gang’ cages. Small amounts of IgG may be transferred from the mother to infant as the result of colostrum and milk; however, the amount of IgG that is transported across the gut to the systemic IgG is minimal. It is our preference and is more physiologically relevant to keep the infant with the mother rather than separating them at birth, and to measure the amount of IgG anti-Ags by obtaining colostrum and milk from the mother after birth. To do this, we remove the infant from the mother for 24 h after birth to obtain colostrum from the mother using a manual breast pump and then return the infant to the mother. The concentrations of the individual baboon immunoglobulin levels are calculated from human IgM and IgG standard curves.

Enzyme immunoassay. Anti-Ags levels are evaluated using a commercially available solid-phase enzyme immunoassay kit (AUSAB-EIA; Abbott Laboratories, Abbott Park, Ill.). All anti-Ags determinations using the commercial enzyme immunoassay are done in duplicate. In this double-sandwich enzyme immunoassay, Ag-coated beads are used to bind anti-Ags present in the serum, and enzyme-labeled Ag serves as the indicator of binding. Based on the individual binding curves generated, we determine the anti-Ags titers based in mIU/ml of sera according to the manufacturers' instructions. Anti-Ags titers greater than 8 mIU/ml are indicative of protective levels of antibodies in humans. We also determine the ratios of the absorbance obtained with the individual sample (S) compared with background negative (N) control. The S/N ratios are included to demonstrate the variability observed between the individual samples.

### Example 3

### Neonatal Induction of Tolerance to Skeletal Tissue Without Immunosuppression

Vascularized allogeneic skeletal tissue transplantation without the need for host immunosuppression would increase reconstructive options for treating congenital and acquired defects. Because the immune system of a fetus or neonate is immature, it may be possible to induce tolerance to allogeneic skeletal tissues by alloantigen injection during this permissive period. Within 12 hours after birth, 17 neonatal Lewis rats are injected through the superficial temporal vein with 3.5 to 5 million human bone marrow cells in 0.1 ml normal saline. Ten weeks after the injection, peripheral blood from the Lewis rats is analyzed for the presence of tolerance to the human marrow cells.

### Example 4

### Harvesting of Tolerized Cells

Hematopoietic stem cells are harvested from the blood of an animal before the start of high-dose chemotherapy in all patients who were to undergo stem-cell transplantation. In the initial stage of the protocol, granulocyte-macrophage colony-stimulating factor was administered to stimulate the mobilization of stem cells from the bone marrow. A minimum of 2×10⁸ nucleated cells per kilogram of body weight is also harvested from the bone marrow and cryopreserved. The bone marrow and blood stem cells are combined and infused after high-dose chemotherapy. If only stem cells from the blood are used, a minimum of 6×10⁸ nucleated cells per kilogram was harvested.

The preparative regimen for stem-cell transplantation lasts four days and consists of a continuous infusion of cyclophosphamide (1500 mg per square meter; total dose, 6000 mg per square meter), carboplatin (200 mg per square meter; total dose, 800 mg per square meter), and thiotepa (125 mg per square meter; total dose, 500 mg per square meter). (10) Stem cells are infused on day 0, approximately 48 hours after the completion of chemotherapy, and granulocyte-macrophage colony-stimulating factor (250 mg per square meter) is administered to stimulate hematopoietic recovery (i.e., until the absolute neutrophil count exceeded 1000 per cubic millimeter for a period of three days).

The animals are killed, and the bone marrow, spleen, and thymus are harvested. Four-color flow cytometric analysis, semi-quantitative PCR, myeloid and erythroid progenitor, and stem cell assays are used to monitor human engraftment. (Transplantation (2000) 15;69(5):927-35)

### Example 5

### Conditioning of Recipient

All patients receive 8 Gy total body irradiation (TBI) in a single dose at a fast dose rate 16 cGy/min midplane) from a 18 MV photon beam linear accelerator on day −5 (5 days prior to engraftment/transplant). Lungs are shielded by individual lead molds; the corrected mean total lung dose was 7 Gy. Thiotepa (Lederle Laboratories, Pearl River, N.Y.) is administered i.v. on day −4 (4 days prior to engraftment) in two divided doses, 5 mg/kg body weight per dose (4 hours for each infusion, total dose 10 mg/kg body weight). On each day from days −4 to −1 (4 to 1 days prior to engraftment/transplant) rabbit anti-human thymocyte globin (ATG; Fresenius, AG Germany) at a dose of 5 mg/kg body weight is infused over 8 hours, followed by cyclophosphamide (Endoxin-Asta, Asta-Werke, Bielefeld, Germany) administered on days −3 and −2 (3 and 2 days prior to engraftment/transplant) at a dose of 60 mg/kg body weight. No immunosuppressive therapy is given as GvHD prophylaxis following transplant.

On day 0 (i.e. 5 days following the irradiation treatment), bone marrow from a tolerized animal, depleted of T-cells by soybean agglutinin and E-rosetting is transplanted into each patient, and preparations of T-cell depleted peripheral blood mononuclear cells (PBMC) from the same donor are administered on days +1 and +2 (i.e. 1 and 2 days after bone marrow transplants; for preparation of the bone marrow and PBMC, see below).

All bone marrow preparations are depleted of T lymphocytes using the soybean agglutination and E-rosetting technique, as previously described (Reisner, Y. et al., (1986) Transplantation 42(3):312-5). This procedure results in a 3-3.5 log.sub.10 reduction in the number of clonable T lymphocytes. Aliquots are taken for differential cell counts, monoclonal antibody (MoAb) staining and GFU-GM assay at each stage of processing. T cell-depleted marrow and peripheral blood cells are frozen in a controlled rate liquid nitrogen freezer and stored in the vapor phase of liquid nitrogen. In some cases, the collections from peripheral blood were performed on the day before and on the day of the transplant; these cells are not cryopreserved.

CFU-GM are measured in whole blood and in the leukapheresis product by plating 0.5.times.10.sup.5 mononuclear cells in a 3% agar solution containing 10% of 5637 cell-line conditioned medium, 20% fetal bovine serum and Iscove medium. Colonies of greater than 40 cells are counted on an inverted microscope (Leica, Wetzlar, Germany) after 10-14 days.

The number of CD34+cells are measured both in whole blood and in the leukapheresis product with a direct immunofluorescence technique using the fluorescein conjugate HPCA-2 monoclonal antibody (Becton Dickinson, Palo Alto, Calif.). Negative control is assessed using a mouse IgGl-FITC. Cells were analyzed on a Profile II (Coulter Corporation, Hialeah, Fla.). A gate is established to include only lymphocytes and mononuclear cells. 10,000 cells were evaluated. The T lymphocytes before and after T cell-depletion are evaluated with an immunocytological technique using an anti-CD3 monoclonal antibody as previously described (Cordell, J. L. et al., 1984).

### Example 6

### Xenotransplantation of Hematopoietic Cells

Subjects are irradiated with x-rays to deplete their immune system, and thereafter received acidified water containing 100 mg/L ciprofloxacin (Bayer AG, Leverkusen, Germany). Test cells are injected intravenously with 106 irradiated (15 Gy) tolerized BM cells as carrier cells within a few hours after the mice are irradiated. The presence of tolerized cells in the BM of human is determined using FACS analysis of cells harvested from the femurs and tibias after first blocking Fc receptors, then by staining with mAb's against CD34 (8G12), CD71 (OKT9), glycophorin A (10F7; kindly provided by P. M. Lansdorp), CD15, CD19, CD20, CD45 (from Becton Dickinson), and CD41a and CD66b (from Pharmacia Biotech, Baie d-Urfe, Quebec, Canada), as described. Levels of nonspecific staining are established by parallel analyses of cells incubated with irrelevant isotype-matched control Ab's labeled with the same fluorochromes. Positive events were counted using gates set to exclude more than 99.99% of events in the negative-control analyses. Poisson statistics and the method of maximum likelihood are used to calculate frequencies of repopulating cells using the L-calc software (StemCell Technologies). Statistical analyses. Comparisons are made using Student's t test.

### Example 7

### Conditions that Enable Human Hematopoietic Stem Cell Engraftment in all NOD-SCID Mice

High marrow seeding efficiency of lymphomyeloid repopulating cells in irradiated subjects is evaluated. Transplantable human hematopoietic stem cells (competitive repopulating units [CRU]) can be quantitated based on their ability to produce large populations of lymphoid and myeloid progeny within 6 weeks in the marrow of intravenously injected, sublethally irradiated subjects (Rice et al., Blood (2000) 96(12):3979-3981).

Cord blood (CB) cells are collected from healthy, full-term infants delivered through cesarean section and are placed in tubes containing heparin. Fetal livers (FL) are removed from 14-to 21-week-old aborted fetuses, using foot-length measurement as a determinant of age, and single-cell suspensions are obtained by first mincing the livers into small fragments and then dissociating these with dispase. For both types of cell samples, approved institutional procedures for obtaining informed consent are observed. Low-density (less than 1.077 g/mL) previously cryopreserved cells, pooled from several CB or FL samples, are washed twice in Iscove medium plus 10% fetal calf serum (StemCell Technologies, Vancouver, BC, Canada) and resuspended either in phosphate-buffered saline for injection into mice or in Iscove medium for colony-forming cell assays.

Competitive repopulating unit assays: CRU assays are performed, and values are calculated as previously reported (Holyoake TL, et al., Exp Hematol. 1999;27:1418-1427; Boggs DR. Am J Hematol. 1984;16:277-286).

What is claimed is:

1. A method for reconstituting a subject's immune system, said method comprising tolerizing the immune system of a non-human animal with antigens from said subject, and thereafter transplanting the bone marrow of said subject with the bone marrow of said tolerized immune system.
